SEQUENCE LISTING (17 sequences)

SEQ ID NO: 1 - protein, 214 aa, Artificial Sequence
  sequence is synthesised
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, Artificial Sequence
  sequence is synthesised
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 4 aa, Artificial Sequence
  antibody variant
CPPS

SEQ ID NO: 4 - protein, 4 aa, Artificial Sequence
  antibody variant
SPPC

SEQ ID NO: 5 - protein, 4 aa, Artificial Sequence
  antibody variant
CPSS

SEQ ID NO: 6 - protein, 4 aa, Artificial Sequence
  antibody variant
SPSC

SEQ ID NO: 7 - protein, 4 aa, Artificial Sequence
  antibody variant
CXXS

SEQ ID NO: 8 - protein, 4 aa, Artificial Sequence
  antibody variant
SXXC

SEQ ID NO: 9 - protein, 7 aa, Artificial Sequence
  antibody variant
CXXXXXS

SEQ ID NO: 10 - protein, 7 aa, Artificial Sequence
  antibody variant
SXXXXXC

SEQ ID NO: 11 - protein, 7 aa, Artificial Sequence
  antibody variant
CPXXXPS

SEQ ID NO: 12 - protein, 7 aa, Artificial Sequence
  antibody variant
SPXXXPC

SEQ ID NO: 13 - protein, 7 aa, Artificial Sequence
  antibody variant
CPPXXXS

SEQ ID NO: 14 - protein, 7 aa, Artificial Sequence
  antibody variant
SPPXXXC

SEQ ID NO: 15 - protein, 7 aa, Artificial Sequence
  antibody variant
CXXXPPS

SEQ ID NO: 16 - protein, 7 aa, Artificial Sequence
  antibody variant
SXXXPPC

SEQ ID NO: 17 - protein, 14 aa, Artificial Sequence
  hinge region of antibody variant
PKSCDKTHTCPPCP

(19) United States
(12) Patent Application Publication
(10) Pub. No.: US 2015/0125473 A1
(43) Pub. Date: May 7, 2015

(54) NOVEL PROCESS FOR PREPARATION OF ANTIBODY CONJUGATES AND NOVEL ANTIBODY CONJUGATES

(71) Applicant: POLYTHERICS LIMITED, London (GB)

(72) Inventors: John Burt, London (GB); Antony Godwin, London (GB); George Badescu, London (GB)

(21) Appl. No.: 14/407,859

(22) PCT Filed: Jun. 19, 2013
(86) PCT No.: PCT/GB2013/051593

§ 371 Date: Dec. 12, 2014

(30) Foreign Application Priority Data
Jun. 19, 2012 (GB) 1210838.7
Apr. 12, 2013 (GB) 1306706.1

(51) Int. Cl. A61K 47/48; A61K 38/06; C07K 16/28

(52) U.S. Cl. CPC A61K 47/48415; C07K 16/2863; A61K 38/06; A61K 47/48646
USPC 424/179.1; 530/391.1; 530/391.5; 530/391.9; 435/69.6

(57) ABSTRACT

The present invention concerns a process for the preparation of an antibody conjugate comprising the step of reacting an engineered antibody having a single inter-heavy chain disulfide bond with a conjugating reagent that forms a bridge between the two cysteine residues derived from the disulfide bond.

This invention relates to a novel process for preparing antibody conjugates, and to novel antibody conjugates.

The specificity of antibodies for specific antigens on the surface of target cells and molecules has led to their extensive use as carriers of a variety of diagnostic and therapeutic agents. For example, antibodies conjugated to labels and reporter groups such as fluorophores, radioisotopes and enzymes find use in labelling and imaging applications, while conjugation to cytotoxic agents and chemotherapy drugs allows targeted delivery of such agents to specific tissues or structures, for example particular cell types or growth factors, minimising the impact on normal, healthy tissue and significantly reducing the side effects associated with chemotherapy treatments. Antibody-drug conjugates have extensive potential therapeutic applications in several disease areas, particularly in cancer.

Previous methods of conjugating a desired moiety to an antibody generally involved non-specific conjugation at sites along an antibody (for example, via lysine side-chain amines), resulting in a heterogeneous distribution of conjugation products and, frequently, unconjugated protein, to give a complex mixture that is difficult and expensive to characterise and purify. Each conjugation product in such a mixture potentially has different pharmacokinetic, distribution, toxicity and efficacy profiles, and non-specific conjugation also frequently results in impaired antibody function.

Conjugation to antibodies can also be carried out via cysteine sulfhydryl groups activated by reducing interchain disulfide bonds, followed by alkylation of each of the free cysteine residues with the moieties to be attached. In an IgG1 monoclonal antibody with four interchain disulfide bonds, this site-specific conjugation leads to a conjugate with up to eight active moieties attached. However, such conjugation methods still produce a heterogeneous mixture of conjugates with variable stoichiometry (0, 2, 4, 6 or 8 moieties per antibody), and with the attached moieties distributed over the eight possible conjugation sites. In addition, during conjugation to these various cysteine residues, the original disulfide bonds cannot always be re-bridged, potentially leading to structural changes and impaired antibody function.

It is important for optimised efficacy and to ensure dose to dose consistency that the number of conjugated moieties per antibody is the same, and that each moiety is specifically conjugated to the same amino acid residue in each antibody. Accordingly, a number of methods have been developed to improve the homogeneity of antibody conjugates.

WO 2006/065533 recognises that the therapeutic index of antibody-drug conjugates can be improved by reducing the drug loading stoichiometry of the antibody below 8 drug molecules/antibody, and discloses engineered antibodies with predetermined sites for stoichiometric drug attachment. The 8 cysteine residues of the parent antibody involved in the formation of interchain disulfide bonds were each systematically replaced with another amino acid residue, to generate antibody variants with either 6, 4 or 2 remaining accessible cysteine residues. Antibody variants with 4 remaining cysteine residues were then used to generate conjugates displaying defined stoichiometry (4 drugs/antibody) and sites of drug attachment, which displayed similar antigen-binding affinity and cytotoxic activity to the more heterogeneous “partially-loaded” 4 drugs/antibody conjugates derived from previous methods.

While the antibodies of WO 2006/065533 generate homogeneous conjugates with improved yield, it is thought that the elimination of the native interchain disulfide bonds could disrupt the quaternary structure of the antibody, thereby perturbing the behaviour of the antibody in vivo, including changes in antibody effector functions (Junutula J R, et al. Nat Biotechnol. 2008 August; 26(8):925-32).

WO 2008/141044 is directed to antibody variants in which one or more amino acids of the antibody is substituted with a cysteine amino acid. The engineered cysteine amino acid residue is a free amino acid and not part of an intrachain or interchain disulfide bond, allowing drugs to be conjugated with defined stoichiometry and without disruption of the native disulfide bonds. There remains, however, a risk that engineering free cysteine residues into the antibody molecule may cause rearrangement and scrambling reactions with existing cysteine residues in the molecule during antibody folding and assembly, or result in dimerisation through reaction with a free cysteine residue in another antibody molecule, leading to impaired antibody function or aggregation.

WO 2005/007197 describes a process for the conjugation of polymers to proteins, using novel conjugation reagents having the ability to conjugate with both sulfur atoms derived from a disulfide bond in a protein to give novel thioether conjugates. In this method, the disulfide bond is reduced to produce two free cysteine residues and then reformed using a bridging reagent to which the polymer is covalently attached, without destroying the tertiary structure or abolishing the biological activity of the protein. This method can however be less efficient for conjugating antibodies than for other proteins, as the relative closeness of neighbouring disulfide bonds in the hinge region of the antibody molecule can result in some disulfide bond scrambling.

We have now found a variation of this process that reduces the problem of disulfide bond scrambling and improves the homogeneity of antibody conjugation during preparation of antibody conjugates.

The present invention therefore provides a process for the preparation of an antibody conjugate comprising the step of reacting an engineered antibody having a single inter-heavy chain disulfide bond with a conjugating reagent that forms a bridge between the two cysteine residues derived from the disulfide bond.

In naturally occurring IgG molecules, the heavy chains of the antibody molecule are linked by multiple interchain disulfide bonds (inter-heavy chain disulfide bonds) between cysteine residues in the hinge region of the antibody. As used herein, an “inter-heavy chain cysteine residue” refers to a cysteine residue of an antibody heavy chain that can be involved in the formation of an inter-heavy chain disulfide bond.

The four IgG subclasses differ with respect to the number of inter-heavy chain disulfide bonds in the hinge region: human IgG1, IgG2, IgG3 and IgG4 isotypes have 2, 4, 11 and 2 inter-heavy chain disulfide bonds, respectively. In IgG1 and IgG4, the heavy chains are linked by disulfide bonds at the hinge region of the antibody between inter-heavy chain cysteine residues at positions corresponding to 226 and 229 according to the EU-index numbering system (Edelman G M, et al., Proc Natl Acad Sci USA. 1969 May; 63(1):78-85). The antibodies used in the present invention have a single inter-heavy chain disulfide bond in the hinge region of the antibody (i.e. generally between positions 221 and 236).

Throughout this specification and claims, the residues in the antibody sequence are conventionally numbered according to the EU-index numbering system. Positions 226 and 229 according to the EU-index numbering system correspond to positions 239 and 242 using the Kabat numbering system (Kabat et al., 1991, Sequences of Proteins of Immunological Interest, 5th Ed., United States Public Health Service, National Institutes of Health, Bethesda) or Chothia numbering system (Al-Lazikani et al., (1997) JMB 273, 927-948). The EU-index residue designations do not always correspond directly with the linear numbering of the amino acid residues in the amino acid sequence. The actual linear amino acid sequence may contain fewer or additional amino acids than in the strict EU-index numbering. The correct EU-index numbering of residues may be determined for a given antibody by alignment of residues of homology in the sequence of the antibody with a “standard” EU-index or Kabat numbered sequence, for example by alignment of residues of the hinge region of the antibody.

The single inter-heavy chain disulfide bond may be either in the location of a disulfide bond in the parent antibody, or it may be in a different location, provided that it is in the hinge region, i.e. the antibody may be engineered to lack all but one of the native hinge disulfide bonds of the parent antibody, or it may be engineered to remove all of the hinge disulfide bonds of the parent antibody, a new disulfide bond being engineered in a new position.

In one embodiment, the process of the invention comprises preparing an engineered antibody having a single inter-heavy chain disulfide bond by recombinant expression or chemical synthesis. For example, one or more inter-heavy chain cysteine residues in a parent antibody sequence can be removed by substitution of cysteine residue(s) with an amino acid other than cysteine, such that the resulting engineered antibody has a single inter-heavy chain cysteine residue in each heavy chain, between which is formed an inter-heavy chain disulfide bond. Alternatively, one or more of the inter-heavy chain cysteine residues in the native parent antibody sequence may be deleted and not replaced by another amino acid. These processes result in an engineered antibody having a single disulfide bond in the same position as a disulfide bond in the parent antibody. If it is desired to introduce a single inter-heavy chain disulfide bond in a different position from a disulfide bond in the parent antibody, then inter-heavy chain cysteine residues in a parent antibody sequence are substituted or deleted, and a new cysteine residue is engineered into the antibody at a different location within the hinge region.

Preferably, the step of preparing an engineered antibody having a single inter-heavy chain disulfide bond comprises
a) mutating a nucleic acid sequence encoding a parent antibody, wherein said mutation results in deletion or substitution of one or more inter-heavy chain cysteine residues with an amino acid other than cysteine;
b) expressing the nucleic acid in an expression system; and
c) isolating the engineered antibody.

Methods for introducing a mutation into a nucleic acid sequence are well known in the art. Such methods include polymerase chain reaction-based mutagenesis, site-directed mutagenesis, gene synthesis using the polymerase chain reaction (PCR) with synthetic DNA oligomers, and nucleic acid synthesis followed by ligation of the synthetic DNA into an expression vector, comprising other portions of the heavy and/or light chain, as applicable (See, e.g., Sambrook et al., Molecular Cloning, A Laboratory Manual, Third Edition, Cold Spring Harbor Publish., Cold Spring Harbor, N.Y. (2001); and Ausubel et al., Current Protocols in Molecular Biology, 4th ed., John Wiley and Sons, New York (1999)). For example, site-directed mutagenesis can be used to substitute one or more inter-heavy chain cysteine residues with an amino acid other than cysteine. Briefly, PCR primer oligonucleotides may be designed to incorporate nucleotide changes into the coding sequence of the subject antibody. For example, a serine substitution mutation can be constructed by designing a primer to change a codon TGT or TGC encoding cysteine to a TCT, TCC, TCA, TCG, AGT or AGC codon encoding serine.

Detailed methods for expressing the antibody-encoding nucleic acid and isolating the antibody from host cell systems are also well known (see, for example, Co et al, J. Immunol, 152:2968-76, 1994; Better and Horwitz, Methods Enzymol., 178:476-96, 1989; Pluckthun and Skerra, Methods Enzymol, 178:497-515, 1989; Lanioyi, Methods Enzymol, 121: 652-63, 1986; Rousseaux et al., Methods Enzymol., 121:663-9, 1986; Bird and Walker, Trends Biotechnol, 9:132-7, 1991). Suitable expressions systems include microorganisms such as bacteria (e.g., E. coli) transformed with recombinant bacteriophage DNA, plasmid DNA or cosmid DNA expression vectors containing antibody coding sequences; yeast (e.g., Saccharomyces; Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, HEK 293, NSO, and 3T3 cells) harbouring recombinant expression constructs containing promoters derived from the genome of mammalian cells (e.g., metallothionein promoter) or from mammalian viruses (e.g., the adenovirus late promoter; the vaccinia virus 7.5K promoter).

It is also contemplated that an antibody having a single inter-heavy chain disulfide bond may be prepared by chemical synthesis using known methods of synthetic protein chemistry. For example, the appropriate amino acid sequence, or portions thereof, may be prepared using peptide synthesis methods well known in the art such as direct peptide synthesis using solid phase techniques (e.g. Merrifield, 1963, J. Am Chem. Soc. 85, 2149; Stewart et al., 1969, in Solid-Phase Peptide Synthesis, WH Freeman Co, San Francisco, Calif.; Matteucci et al. J. Am. Chem. Soc. 103:3185-3191, 1981) or automated synthesis, for example using a Synthesiser from Applied Biosystems (California, USA). Various portions of the antibody may also be synthesised separately, for example, antibody fragments may be derived via proteolytic digestion of intact antibodies (Morimoto et al (1992) Journal of Biochemical and Biophysical Methods 24:107-117; and Brennan et al (1985) Science, 229:81), produced directly by recombinant host cells, or isolated from the antibody phage libraries, and combined using chemical coupling methods to produce the desired antibody molecule.

Preferably, said single inter-heavy chain disulfide bond is at position 226 or 229 of the antibody according to the EU-index numbering system (position 239 or 242 using the Kabat numbering system).

Preferably, the antibody has an amino acid other than cysteine at position 226 or 229 according to the EU-index numbering system. For example, the native cysteine residue at position 226 or 229 can be substituted for an amino acid other than cysteine. An amino acid substituted for the native cysteine residue at position 226 or 229 should not include a thiol moiety, and may be serine, threonine, valine, alanine, glycine, leucine or isoleucine, other polar amino acid, other naturally occurring amino acid, or non-naturally occurring amino acid. Preferably, the cysteine residue at position 226 or 229 is substituted with serine.

In one embodiment, the antibody has a cysteine at position 226 and an amino acid other than cysteine at position 229, for example serine. In another embodiment, the antibody has an amino acid other than cysteine at position 226, for example serine, and a cysteine at position 229.

For example, the antibody may be an IgG1 molecule and comprise a sequence of Cys-Pro-Pro-Ser or Ser-Pro-Pro-Cys at positions 226-229 according to the EU-index numbering system; that is to say that the sequence between 226 and 229 is wild type. Alternatively, the antibody may be an IgG4 molecule and comprise a sequence of Cys-Pro-Ser-Ser or Ser-Pro-Ser-Cys at positions 226-229 according to the EU-index numbering system; that is to say that the sequence between 226 and 229 is wild type.

Alternatively, the sequence between residues 226 and 229 may contain mutations from wild type. For example in an IgG4 may be Cys-Pro-Pro-Ser, and Ser-Pro-Pro-Cys. More generally, the sequence in an IgG1 or an IgG4 may be Cys-(Xaa)-(Xaa)-Ser or Ser-(Xaa)-(Xaa)-Cys, where each Xaa is independently any amino acid lacking a thiol moiety. For example, each Xaa can independently be an amino acid selected from serine, threonine, valine, alanine, glycine, leucine or isoleucine, other polar amino acid, other naturally occurring amino acid, or non-naturally occurring amino acid. For example, each Xaa can be selected from serine, threonine and valine, for example serine.

In a further alternative, there may be more than two amino acid residues between residues 226 and 229. For example, the sequence may be Cys-(Xaa)ₙ-Ser or Ser-(Xaa)ₙ-Cys, where n is 3, 4 or 5 and each Xaa is independently any amino acid lacking a thiol moiety. There may be specifically mentioned Cys-Pro-(Xaa)ₘ-Pro-Ser, Ser-Pro-(Xaa)ₘ-Pro-Cys, Cys-Pro-Pro-(Xaa)ₘ-Ser, Ser-Pro-Pro-(Xaa)ₘ-Cys, Cys-(Xaa)ₘ-Pro-Pro-Ser and Ser-(Xaa)ₘ-Pro-Pro-Cys, where m is 1, 2 or 3, and each Xaa is independently any amino acid lacking a thiol moiety. For example, each Xaa can independently be an amino acid selected from serine, threonine, valine, alanine, glycine, leucine or isoleucine, other polar amino acid, other naturally occurring amino acid, or non-naturally occurring amino acid. For example, each Xaa can be selected from serine, threonine and valine, for example serine.

Throughout this specification, the term “antibody” should be understood to mean an immunoglobulin molecule that recognises and specifically binds to a target antigen, such as a protein, polypeptide, peptide, carbohydrate, polynucleotide, lipid, or combination thereof through at least one antigen recognition site within the variable region of the immunoglobulin molecule. The term “antibody” encompasses polyclonal antibodies, monoclonal antibodies, multispecific antibodies such as bispecific antibodies, chimeric antibodies, humanised antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. The use of IgG1 or IgG4 is particularly preferred.

Throughout this specification and claims, except where the context requires otherwise, the term “antibody” encompasses full length antibodies and antibody fragments comprising an antigen-binding region of the full length antibody and a single inter-heavy chain disulfide bond. The antibody fragment may for example be F(ab′)₂ or multispecific antibodies formed from antibody fragments, for example minibodies composed of different permutations of scFv fragments or diabodies and Fc fragments or C_{H} domains such as scFv-Fc, scFv-Fc-scFv, (Fab′ScFv)₂, scDiabody-Fc, scDiabody-C_{H}3, scFv-C_{H}3, scFv-C_{H}2-C_{H}3 fusion proteins and so forth. An antibody fragment can be produced by enzymatic cleavage, synthetic or recombinant techniques discussed above.

Preferably, the antibody conjugates find use in clinical medicine for diagnostic and therapeutic purposes. For example, the conjugating reagent may comprise a diagnostic or therapeutic agent, or a binding agent capable of binding a diagnostic or therapeutic agent. Such conjugates find use in therapy, for example for the treatment of cancer, or for in vitro or in vivo diagnostic applications. The antibody conjugates may also be used in non-clinical applications. For example, the conjugating reagent may comprise a labelling agent or a binding agent capable of binding a labelling agent, for example for use in immunoassays to detect the presence of a particular antigen or applications such as fluorescence activated cell sorting (FACS) analysis.

A wide variety of diagnostic, therapeutic and labelling agents that are known in the art have been conjugated to antibody molecules. For example, the conjugating agent may include a diagnostic agent, a drug molecule, for example a cytotoxic agent, a toxin, a radionuclide, a fluorescent agent (for example an amine derivatised fluorescent probe such as 5-dimethylaminonaphthalene-1-(N-(2-aminoethyl))sulfonamide-dansyl ethylenediamine, Oregon Green® 488 cadaverine (catalogue number 0-10465, Molecular Probes), dansyl cadaverine, N-(2-aminoethyl)-4-amino-3,6-disulfo-1,8-naphthalimide, dipotassium salt (lucifer yellow ethylenediamine), or rhodamine B ethylenediamine (catalogue number L-2424, Molecular Probes), or a thiol derivatised fluorescent probe for example BODIPY® FL L-cystine (catalogue number B-20340, Molecular Probes); or a binding agent, for example a chelating agent, which could then be used to bind, e.g. chelate, any other desired moiety, for example one of those mentioned above.

The conjugating reagent may also include an oligomer or a polymer (jointly referred to herein as “polymer” for convenience). Water soluble, synthetic polymers, particularly polyalkylene glycols, are widely used to conjugate therapeutically active molecules such as proteins, including antibodies. These therapeutic conjugates have been shown to alter pharmacokinetics favourably by prolonging circulation time and decreasing clearance rates, decreasing systemic toxicity, and in several cases, displaying increased clinical efficacy. The process of covalently conjugating polyethylene glycol, PEG, to proteins is commonly known as “PEGylation”.

A polymer may for example be a polyalkylene glycol, a polyvinylpyrrolidone, a polyacrylate, for example polyacryloyl morpholine, a polymethacrylate, a polyoxazoline, a polyvinylalcohol, a polyacrylamide or polymethacrylamide, for example polycarboxymethacrylamide, or a HPMA copolymer. Additionally, the polymer may be a polymer that is susceptible to enzymatic or hydrolytic degradation. Such polymers, for example, include polyesters, polyacetals, poly(ortho esters), polycarbonates, poly(imino carbonates), and polyamides, such as poly(amino acids). A polymer may be a homopolymer, random copolymer or a structurally defined copolymer such as a block copolymer, for example it may be a block copolymer derived from two or more alkylene oxides, or from poly(alkylene oxide) and either a polyester, polyacetal, poly(ortho ester), or a poly(amino acid). Polyfunctional polymers that may be used include copolymers of divinylether-maleic anhydride and styrene-maleic anhydride.

Naturally occurring polymers may also be used, for example polysaccharides such as chitin, dextran, dextrin, chitosan, starch, cellulose, glycogen, poly(sialylic acid), hyaluronic acid and derivatives thereof. A protein may be used as the polymer. This allows conjugation to the antibody or antibody fragment, of a second protein, for example an enzyme or other active protein, or a scaffolding protein such as avidin that can bind to biotinylated molecules. Also, if a peptide containing a catalytic sequence is used, for example an O-glycan acceptor site for glycosyltransferase, it allows the incorporation of a substrate or a target for subsequent enzymatic reaction. Polymers such as polyglutamic acid may also be used, as may hybrid polymers derived from natural monomers such as saccharides or amino acids and synthetic monomers such as ethylene oxide or methacrylic acid.

If the polymer is a polyalkylene glycol, this is preferably one containing C₂ and/or C₃ units, and is especially a polyethylene glycol. A polymer, particularly a polyalkylene glycol, may contain a single linear chain, or it may have branched morphology composed of many chains either small or large. The so-called Pluronics are an important class of PEG block copolymers. These are derived from ethylene oxide and propylene oxide blocks. Substituted, or capped, polyalkylene glycols, for example methoxypolyethylene glycol, may be used.

The polymer may, for example, be a comb polymer produced by the method described in WO 2004/113394, the contents of which are incorporated herein by reference. For example, the polymer may be a comb polymer having a general formula:

A-(D)_{d}-(E)ₑ-(F)_{f}

where:
A may or may not be present and is a moiety capable of binding to a protein or a polypeptide;
D, where present, is obtainable by additional polymerisation of one or more olefinically unsaturated monomers which are not as defined in E;
E is obtainable by additional polymerisation of a plurality of monomers which are linear, branched, or star-shaped substituted or non-substituted, and have an olefinically unsaturated moiety;
F, where present, is obtainable by additional polymerisation of one or more olefinically-unsaturated monomers which are not as defined in E;
d and f are an integer between 0 and 500;
e is an integer of 0 to 1000;

wherein when A is present, at least one of D, E and F is present.

The polymer may optionally be derivatised or functionalised in any desired way. In one preferred embodiment, the polymer carries a diagnostic agent, a therapeutic agent, or a labelling agent, for example one of those mentioned above, or a binding agent capable of binding a diagnostic agent, a therapeutic agent, or a labelling agent. Reactive groups may be linked at the polymer terminus or end group, or along the polymer chain through pendent linkers; in such case, the polymer is for example a polyacrylamide, polymethacrylamide, polyacrylate, polymethacrylate, or a maleic anhydride copolymer. Multimeric conjugates that contain more than one biological molecule, can result in synergistic and additive benefits. If desired, the polymer may be coupled to a solid support using conventional methods.

The optimum molecular weight of the polymer will of course depend upon the intended application. Long-chain polymers may be used, for example the number average molecular weight may be in the range of from 500 g/mole to around 75,000 g/mole. However, very small oligomers, consisting for example of as few as 2 repeat units, for example from 2 to 20 repeat units, are useful for some applications. When the antibody conjugate is intended to leave the circulation and penetrate tissue, for example for use in the treatment of inflammation caused by malignancy, infection or autoimmune disease, or by trauma, it may be advantageous to use a lower molecular weight polymer in the range up to 30,000 g/mole. For applications where the antibody conjugate is intended to remain in circulation it may be advantageous to use a higher molecular weight polymer, for example in the range of 20,000-75,000 g/mole.

The polymer to be used should be selected so the conjugate is soluble in the solvent medium for its intended use. For biological applications, particularly for diagnostic applications and therapeutic applications for clinical therapeutic administration to a mammal, the conjugate will be soluble in aqueous media.

Preferably the polymer is a synthetic polymer, and preferably it is a water-soluble polymer. The use of a water-soluble polyethylene glycol is particularly preferred for many applications.

Any suitable conjugating reagent that is capable of reacting with the antibody via both the thiol groups produced by reduction of the disulfide bond may be used.

One group of reagents are bis-halo- or bis-thio-maleimides and derivatives thereof as described in Smith et al, J. Am. Chem. Soc. 2010, 132, 1960-1965, and Schumaker et al, Bioconj. Chem., 2011, 22, 132-136. These reagents contain the functional grouping:

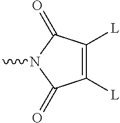

in which each L is a leaving group, for example one of those mentioned below. Preferred leaving groups include halogen atoms, for example chlorine, bromine or iodine atoms, —S.CH₂CH₂OH groups, and —S-phenyl groups. The nitrogen atom of the maleimide ring may carry a diagnostic, therapeutic or labelling agent, or a binding agent for a diagnostic, therapeutic or labelling agent, for example one of the formula D-Q- mentioned below.

In a preferred embodiment of the invention, the reagent contains the functional group:

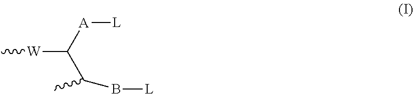

in which W represents an electron-withdrawing group, for example a keto group, an ester group —O—CO—, a sulfone group —SO₂—, or a cyano group; A represents a C₁₋₅ alkylene or alkenylene chain; B represents a bond or a C₁₋₄ alkylene or alkenylene chain; and each L independently represents a leaving group. Reagents of this type are described in Bioconj. Chem 1990(1), 36-50, Bioconj. Chem 1990(1), 51-59, and J. Am. Chem. Soc. 110, 5211-5212. Preferred meanings for W, A, B and L are as given below.

Such reagents may carry a diagnostic, therapeutic or labelling agent, or a binding agent for a diagnostic, therapeutic or labelling agent. In this case, the reagents may have the formula (Ia) or, where W represents a cyano group, (Ib):

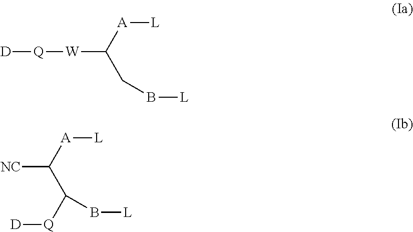

in which Q represents a linking group and D represents a diagnostic, therapeutic or labelling agent, or a binding agent for a diagnostic, therapeutic or labelling agent. Preferred groups Q are given below for the formulae II, III and IV.

A particularly preferred functional group of this type has the formula:

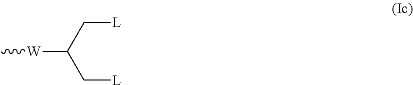

For example, the group may be of the formula:

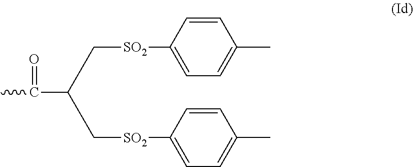

When such a reagent may carries a diagnostic, therapeutic or labelling agent, or a binding agent for a diagnostic, therapeutic or labelling agent, it has the formula:

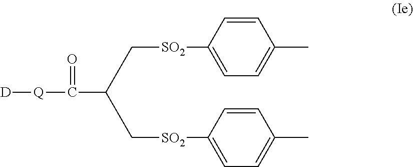

in which Q and D have the meanings given above. Preferred groups Q are given below for the formulae II, III and IV.

A particularly preferred reagent of this type has the formula:

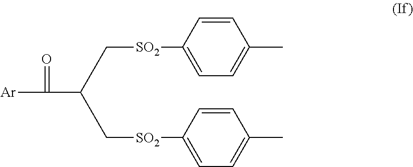

in which Ar represents an optionally-substituted phenyl group, for example one of those listed below for the compounds of the formulae II, III and IV. For example, the reagent, or a precursor of the reagent, may be of the formula:

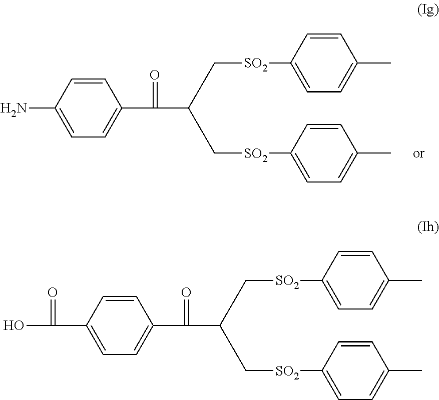

The above reagents may be functionalised to carry a diagnostic, therapeutic or labelling agent, or a binding agent for a diagnostic, therapeutic or labelling agent. For example, the NH₂ group shown in the formulae (Ig) or the carboxylic acid group in formula (Ih) above may be used to react with any suitable group in order to attach a diagnostic, therapeutic or labelling agent, or a binding group for a diagnostic, therapeutic or labelling agent, giving a compound of the formulae (Ig) or (Ih) in which the NH₂ group or carboxylic acid group is replaced by a group D-Q-; or the phenyl group in the formulae (If), (Ig) or (Ih) above may carry a suitable reactive group.

When the conjugating reagent comprises a polymer, the reagent may be one of the reagents described in WO 99/45964, WO 2005/007197, or WO 2010/100430, the contents of which are incorporated herein by reference. Preferably a polymer-containing reagent contains a functional group I as described above and is of the formula II, III or IV below:

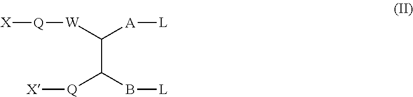

in which one of X and X′ represents a polymer and the other represents a hydrogen atom;
Q represents a linking group;
W represents an electron-withdrawing group, for example a keto group, an ester group —O—CO— or a sulfone group —SO₂—; or, if X′ represents a polymer, X-Q-W together may represent an electron withdrawing group;
A represents a C₁₋₅ alkylene or alkenylene chain;
B represents a bond or a C₁₋₄ alkylene or alkenylene chain; and
each L independently represents a leaving group;

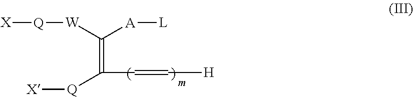

in which X, X′, Q, W, A and L have the meanings given for the general formula II, and in addition if X represents a polymer, X′ and electron-withdrawing group W together with the interjacent atoms may form a ring, and m represents an integer 1 to 4; or

X-Q-W—CR¹R^{1′}—CR².L.L′ (IV)

in which X, Q and W have the meanings given for the general formula II, and either
R¹ represents a hydrogen atom or a C₁₋₄alkyl group, R¹ represents a hydrogen atom, and each of L and L′ independently represents a leaving group; or
R¹ represents a hydrogen atom or a C₁₋₄alkyl group, L represents a leaving group, and R^{1′} and L′ together represent a bond; or

R¹ and L together represent a bond and R^{1′} and L′ together represent a bond; and R² represents a hydrogen atom or a C₁₋₄ alkyl group.

A linking group Q may for example be a direct bond, an alkylene group (preferably a C₁₋₁₀ alkylene group), or an optionally-substituted aryl or heteroaryl group, any of which may be terminated or interrupted by one or more oxygen atoms, sulfur atoms, —NR groups (in which R represents a hydrogen atom or an alkyl (preferably C₁₋₆alkyl), aryl (preferably phenyl), or alkyl-aryl (preferably C₁₋₆alkyl-phenyl) group), keto groups, —O—CO— groups, —CO—O— groups, —O—CO—O, —O—CO—NR—, —CO—NR— and/or —NR.CO— groups. Such aryl and heteroaryl groups Q form one preferred embodiment of the invention. Suitable aryl groups include phenyl and naphthyl groups, while suitable heteroaryl groups include pyridine, pyrrole, furan, pyran, imidazole, pyrazole, oxazole, pyridazine, primidine and purine.

Especially suitable linking groups Q are heteroaryl or, especially, aryl groups, especially phenyl groups, terminated adjacent the polymer X by an —NR.CO— group. The linkage to the polymer may be by way of a hydrolytically labile bond, or by a non-labile bond. W may for example represent a keto group CO, an ester group —O—CO— or a sulfone group —SO₂—; or, if X-Q-W— together represent an electron withdrawing group, this group may for example be a cyano group. Preferably X represent a polymer, and X′-Q- represents a hydrogen atom.

Substituents which may be present on an optionally substituted aryl or heteroaryl group include for example one or more of the same or different substituents selected from alkyl (preferably C₁₋₄alkyl, especially methyl, optionally substituted by OH or CO₂H), —CN, —NO₂, —CO₂R, —COH, —CH₂OH, —COR, —OR, —OCOR, —OCO₂R, —SR, —SOR, —SO₂R, —NHCOR, —NRCOR, NHCO₂R, —NR.CO₂R, —NO, —NHOH, —NR.OH, —C═N—NHCOR, —C═N—NR.COR, —N⁺R₃, —N⁺H₃, —N⁺HR₂, —N⁺H₂R, halogen, for example fluorine or chlorine, —C≡CR, —C═CR₂ and —C═CHR, in which each R independently represents a hydrogen atom or an alkyl (preferably C₁₋₆alkyl), aryl (preferably phenyl), or alkyl-aryl (preferably C₁₋₆alkyl-phenyl) group. The presence of electron withdrawing substituents is especially preferred. Preferred substituents include for example CN, NO₂, —OR, —OCOR, —SR, —NHCOR, —NR.COR, —NHOH and —NR.COR.

A leaving group L may for example represent —SR, —SO₂R, —OSO₂R, —N⁺R₃, —N⁺HR₂, —N⁺H₂R, halogen, or —OØ, in which R has the meaning given above, and Ø represents a substituted aryl, especially phenyl, group, containing at least one electron withdrawing substituent, for example —CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NR′CO₂R, —NO, —NHOH, —NR′OH, —C═N—NHCOR, —C═N—NR′COR, —N⁺R₃, —N⁺HR₂, —N⁺H₂R, halogen, especially chlorine or, especially, fluorine, —C≡CR, —C═CR₂ and —C═CHR, in which each R independently has one of the meanings given above.

An especially preferred polymeric conjugation reagent has the formula:

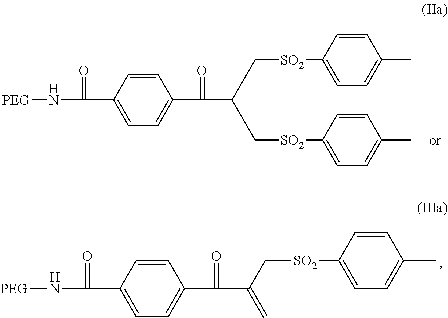

in which the PEG may optionally carry a diagnostic agent, a therapeutic agent, or a labelling agent, for example one of those mentioned above, or a binding agent capable of binding a diagnostic agent, a therapeutic agent, or a labelling agent.

The immediate product of the conjugation process using one of the reagents described above is a conjugate which contains an electron-withdrawing group W. However, the process of the invention is reversible under suitable conditions. This may be desirable for some applications, for example where rapid release of the antibody is required, but for other applications, rapid release of the antibody may be undesirable. It may therefore be desirable to stabilise the conjugates by reduction of the electron-withdrawing moiety W to give a moiety which prevents release of the protein. Accordingly, the process described above may comprise an additional optional step of reducing the electron withdrawing group W in the conjugate. The use of a borohydride, for example sodium borohydride, sodium cyanoborohydride, potassium borohydride or sodium triacetoxyborohydride, as reducing agent is particularly preferred. Other reducing agents which may be used include for example tin(II) chloride, alkoxides such as aluminium alkoxide, and lithium aluminium hydride.

Thus, for example, a moiety W containing a keto group may be reduced to a moiety containing a CH(OH) group; an ether group CH.OR may be obtained by the reaction of a hydroxy group with an etherifying agent; an ester group CH.O.C(O)R may be obtained by the reaction of a hydroxy group with an acylating agent; an amine group CH.NH₂, CH.NHR or CH.NR₂ may be prepared from a ketone by reductive amination; or an amide CH.NHC(O)R or CH.N(C(O)R)₂ may be formed by acylation of an amine. A sulfone may be reduced to a sulfoxide, sulfide or thiol ether. A cyano group may be reduced to an amine group.

A key feature of using conjugation reagents described above is that an a-methylene leaving group and a double bond are cross-conjugated with an electron withdrawing function that serves as a Michael activating moiety. If the leaving group is prone to elimination in the cross-functional reagent rather than to direct displacement and the electron-withdrawing group is a suitable activating moiety for the Michael reaction then sequential intramolecular bis-alkylation can occur by consecutive Michael and retro Michael reactions. The leaving moiety serves to mask a latent conjugated double bond that is not exposed until after the first alkylation has occurred and bis-alkylation results from sequential and interactive Michael and retro-Michael reactions. The electron withdrawing group and the leaving group are optimally selected so bis-alkylation can occur by sequential Michael and retro-Michael reactions. It is also possible to prepare cross-functional alkylating agents with additional multiple bonds conjugated to the double bond or between the leaving group and the electron withdrawing group.

Generally, reaction of the antibody with the conjugating reagent involves reducing the hinge disulfide bond in the antibody and subsequently reacting the reduced product with the conjugating reagent. Suitable reaction conditions are given in the references mentioned above. The process may for example be carried out in a solvent or solvent mixture in which all reactants are soluble. The antibody may be allowed to react directly with the conjugation reagent in an aqueous reaction medium. This reaction medium may also be buffered, depending on the pH requirements of the nucleophile. The optimum pH for the reaction will generally be at least 4.5, typically between about 5.0 and about 8.5, preferably about 5.0 to 7.5. The optimal reaction conditions will of course depend upon the specific reactants employed.

Reaction temperatures between 3-37° C. are generally suitable. Reactions conducted in organic media (for example THF, ethyl acetate, acetone) are typically conducted at temperatures up to ambient.

The antibody can be effectively conjugated with the desired reagent using a stoichiometric equivalent or an excess of reagent. The reagent may, for example, be used in a stoichiometric ratio of reagent to number of inter-chain disulfide bonds of the antibody. For example, the reagent may be used in an amount of 0.25 to 4 equivalents, for example between 0.5 to 2 equivalents or between 0.5 to 1.5 equivalents per inter-chain disulfide bond of the antibody. The reagent may, for example, be used in an amount of about 1 equivalent per inter-chain disulfide bond of the antibody. Excess reagent and the product can be easily separated during routine purification, for example by standard chromatography methods, e.g. ion exchange chromatography or size exclusion chromatography, diafiltration, or, when a polyhistidine tag is present, by separation using metal affinity chromatography, e.g. based on nickel or zinc.

While the conjugation reagents of the formulae II, III and IV as shown above contain a polymer, the person skilled in the art would recognise that the discussion above is equally applicable for conjugation of any diagnostic, therapeutic or labelling agent to an antibody in accordance with the process of the invention using reagents containing the functional group I.

The process of the present invention allows an antibody to be effectively conjugated with 1, 2, or 3 conjugating reagents, i.e., across the single inter-heavy chain disulfide bond in the hinge region of the antibody and across the interchain disulfide bonds located between the C_{L} domain of the light chain and the C_{H}1 domain of the heavy chain of the antibody. Preferred conjugates according to the invention comprise 3 conjugated molecules per antibody. Especially preferred conjugates comprise 3 conjugated drug or diagnostic molecules per antibody. The drug/diagnostic agent may be conjugated directly to the antibody by using a conjugating reagent already carrying the drug/diagnostic agent, or the drug/diagnostic agent may be added after conjugation of the conjugating reagent with the antibody, for example by use of a conjugating reagent containing a binding group for the drug/diagnostic agent.

The process of the invention thus allows antibody conjugates to be produced with improved homogeneity. In particular, the use of conjugating reagents that bind across the interchain disulfide bonds of the antibody provides antibody conjugates having improved loading stoichiometry, and in which there are specific sites of attachment, without destroying the native interchain disulfide bonds of the antibody. Bridging of the native disulfide bonds by the conjugating agents thus improves the stability to the antibody conjugate and retains antibody binding and function. The use of antibodies having a single inter-heavy chain disulfide bond, for example at either position 226 or 229, also reduces disulfide bond scrambling. Disulfide scrambling, that is, the incorrect assembly of the cysteine pairs into disulfide bonds, is known to affect the antigen-binding capacity of an antibody and lead to reduced activity. Minimising scrambling by use of the present invention improves the homogeneity of the conjugated antibody.

Antibody-drug conjugates with improved homogeneity provide benefits in therapy, for example a higher therapeutic index, improving efficacy and reducing toxicity of the drug. Homogeneous antibody conjugates also provide more accurate and consistent measurements in diagnostic and imaging applications.

The process of the invention also allows antibody conjugates to be produced with a lower level of drug loading, i.e., a lower drug to antibody ratio (DAR), without disruption of the quaternary structure of the antibody. Although antibody-drug conjugate potency in vitro has been shown to be directly dependent on drug loading (Hamblett K J, et al., Clin Cancer Res. 2004 Oct. 15; 10(20):7063-70) in-vivo antitumour activity of antibody-drug conjugates with four drugs per molecule (DAR 4) was comparable with conjugates with eight drugs per molecule (DAR 8) at equal mAb doses, even though the conjugates contained half the amount of drug per mAb. Drug-loading also affected plasma clearance, with the DAR 8 conjugate being cleared 3-fold faster than the DAR 4 conjugate and 5-fold faster than a DAR 2 conjugate. To maximise the therapeutic potential of antibody-drug conjugates a high therapeutic index is needed, and thus increases in therapeutic index without reduction in efficacy should lead to improved therapies (Hamblett K J, et al., 2004).

The antibody conjugates prepared by the process of the present invention are novel, and the invention therefore provides these conjugates per se, as well as an antibody conjugate prepared by the process of the invention. The invention further provides a pharmaceutical composition comprising such an antibody conjugate together with a pharmaceutically acceptable carrier, optionally together with an additional therapeutic agent; such a conjugate for use as a medicament, especially, where the conjugating agent includes a cytotoxic agent, as a medicament for the treatment of cancer; and a method of treating a patient which comprises administering a pharmaceutically-effective amount of such a conjugate or pharmaceutical composition to a patient.

The invention will now be described by way of example with reference to the drawings, in which:

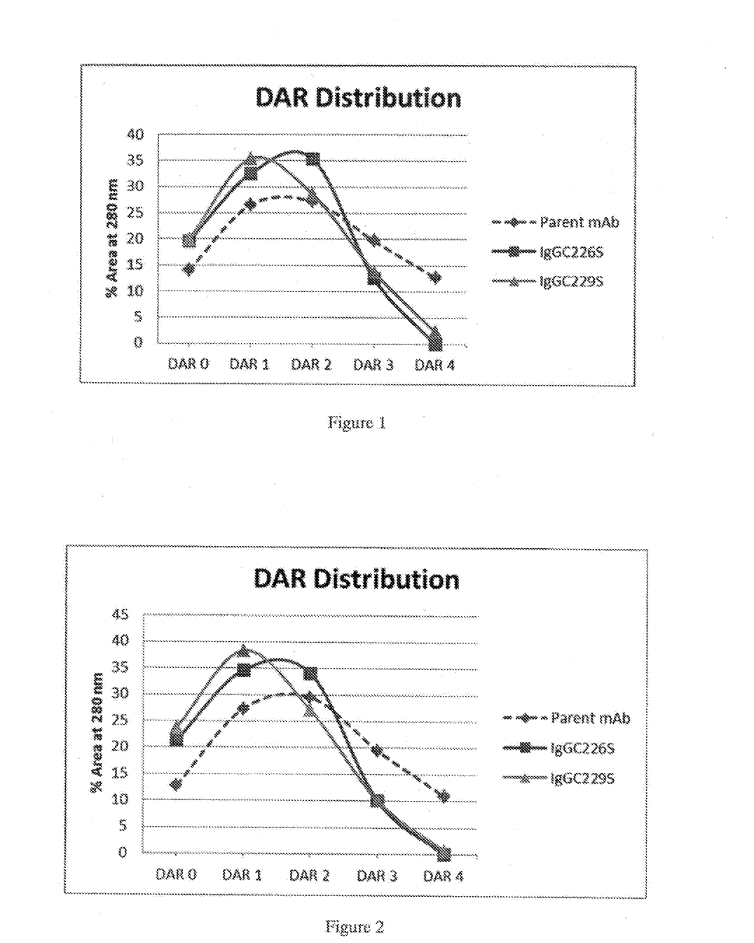

FIG. 1 shows a graph of drug-antibody ratio (DAR) distribution for conjugation reactions carried out at 40° C., using a polymeric conjugation reagent and i) a parent antibody (“parent mAb”), ii) an engineered antibody having a single hinge disulfide bond at position 229 (“IgGC226S”), and iii) an engineered antibody having a single hinge disulfide bond at position 226 (“IgGC229S”).

FIG. 2 shows a graph of drug-antibody ratio (DAR) distribution for conjugation reactions carried out at 22° C., using a polymeric conjugation reagent and i) a parent antibody (“parent mAb”), ii) an engineered antibody having a single hinge disulfide bond at position 229 (“IgGC226S”), and iii) an engineered antibody having a single hinge disulfide bond at position 226 (“IgGC229S”).

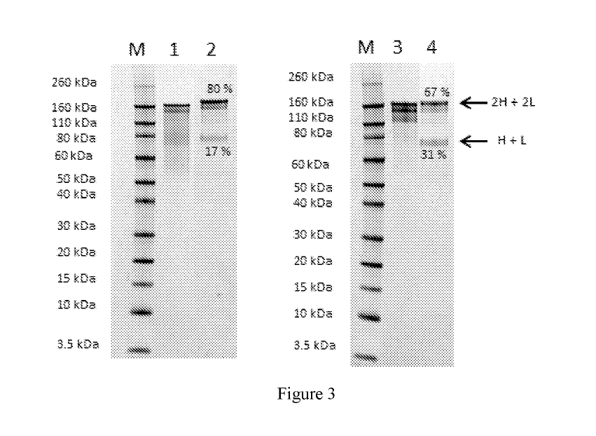

FIG. 3 shows SDS-PAGE analysis of parent antibody and antibody variant IgGC226S pre- and post-conjugation with a polymeric conjugation reagent.

### EXAMPLE 1

### Preparation of Variant Antibody-Drug Conjugates

Two engineered antibody variants, each having a single inter-heavy chain disulfide bond, were created by PCR-based site-directed mutagenesis of the parent antibody sequence in order to demonstrate that the process of the invention allows antibody conjugates to be produced at high levels of homogeneity and with a low average DAR. These antibody variants and the parent antibody were then reacted with a conjugating reagent (Bis-sulfone-PEG(24)-val-cit-PAB-MMAE) that forms a bridge between the two cysteine residues derived from a disulfide bond.

Synthesis of Valine-Citroline-Paraaminobenzyl-Monomethyl Auristatin E (val-cit-PAB-MMAE) Reagent 1 Possessing a 24 Repeat Unit PEG with Terminal Bis-Sulfone Functionality.

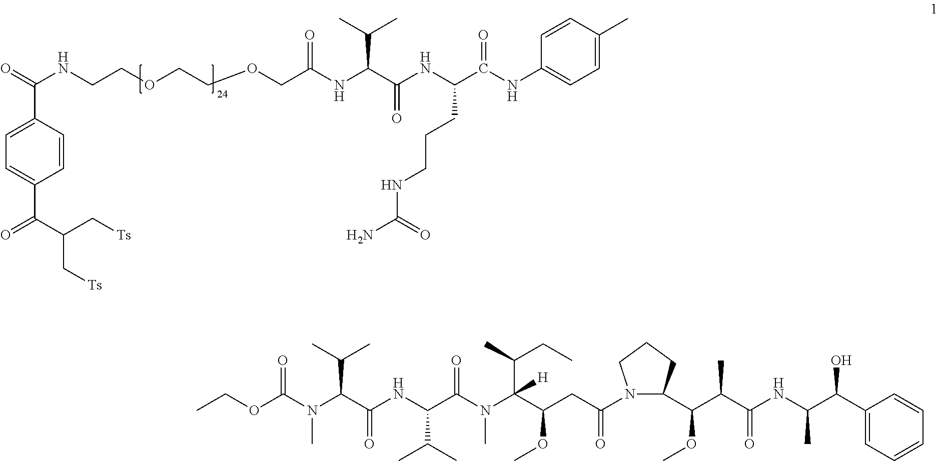

### Step 1: Conjugation of 4-[2,2-bis[(p-tolylsulfonyl)-methyl]acetyl]benzoic acid-N-hydroxy succinimidyl ester (bis-sulfone) to H₂N-dPEG(24)-CO—OtBu

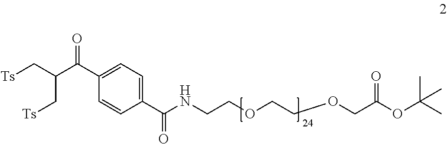

A toluene (3 mL) solution of H₂N-dPEG(24)-CO—OtBu (1.057 g, Iris Biotech) was evaporated to dryness and the residue re-dissolved in dichloromethane (25 mL). Under stirring, 4-[2,2-bis[(p-tolylsulfonyl)-methyl]acetyl]benzoic acid-N-hydroxy succinimidyl ester (1.0 g; Nature Protocols, 2006, 1(54), 2241-2252) was added and the resulting solution further stirred for 72 h at room temperature under an argon atmosphere. Volatiles were removed in vacuo and the solid residue was dissolved in warm acetone (30 mL) and filtered through non-absorbent cotton wool. The filtrate was cooled to −80° C. to precipitate a solid which was isolated by centrifugation at −9° C., for 30 min at 4000 rpm. The supernatant was removed and the precipitation/isolation process repeated 2 additional times. Finally the supernatant was removed and the resulting solid was dried in vacuo to give the bis-sulfone as a colourless amorphous solid (976 mg, 68%). ¹HNMR ∂_{H} (400 MHz CDCl₃)1.45 (9H, s, O′Bu), 2.40-2.45 (8H, m, Ts-Me and CH₂COO′Bu), 3.40-3.46 (2H, m, CH₂-Ts), 3.52-3.66 (m, PEG and CH₂-Ts), 4.27 (1H, q, J 6.3, CH—COAr), 7.30 (4H, d, J 8.3, Ts), 7.58 (2H, d, J 8.6, Ar), 7.63 (4H, d, J 8.3, Ts), 7.75 (2H, d, J 8.6, Ar).

### Step 2. Removal of the Tert-Butyl Protection Group

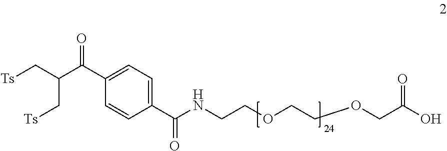

To a stirred solution of the product of step 1 (976 mg) in dichloromethane (4 mL) was added trifluoroacetic acid (4 mL) and the resulting solution was stirred for a further 2 h. Volatiles were then removed in vacuo and the residue was dissolved in warm acetone (30 mL). The product was isolated by precipitation from acetone as described in step 1 to give afford the product 2 as a white powder (816 mg, 85%). ¹HNMR ∂_{H} (400 MHz CDCl₃) 2.42 (6H, s, Ts-Me), 2.52 (2H, t, J 6.1, CH₂—COOH), 3.42 (4H, dd, J 6.3 & 14.5, CH₂-Ts), 3.50-3.64 (m, PEG), 3.68-3.73 (4H, m, PEG), 4.23-4.31 (1H, m, CH—COAr), 7.29 (2H, d, J 8.1, Ar), 7.55-7.65 (6H, m, Ar and Ts), 7.77 (2H, d, J 8.2, Ar)

### Step 3: Conjugation of H₂N-val-cit-PAB-MMAE to Acid Terminated PEGylated bis-sulfone 2

N-methyl morpholine (7.5 mg) was added to a stirred solution of bis-sulfone-PEG-COOH (45 mg) and HATU (13 mg) in dichloromethane-dimethylformamide (85:15 v/v, 6 mL). After stirring for 30 min at room temperature, the H₂N-val-cit-PAB-MMAE (38 mg, Concortis, prepared as in WO 2005/081711) was added and the mixture further stirred for 24 h at room temperature. The reaction mixture was diluted with dichloromethane and washed with 1 M HCl, aqueous NaHCO₃ 10% w/v, brine and then dried with MgSO₄. The crude material was further purified by column chromatography eluting with dichloromethane-methanol (90:10 v/v), the solvent was removed under vacuum and the bis-sulfone-PEG(24)-MMAE product 1 was isolated as a transparent colourless solid (31 mg, 41%) m/zM+Na 2758.5; diagnostic signals for ¹HNMR ∂_{H} (400 MHz CDCl₃)0.60-0.99 (m, aliphatic side chains), 2.43 (s, Me-Ts), 3.36-3.66 (m, PEG), 7.15-7.28 (m, Ar), 7.31 (d, J 8.3, Ar), 7.54-7.62 (m, Ar), 7.79 (d, J 8.3, Ar).

Preparation of the Parent Antibody and Antibody Variants (IgGC226S and IgGC229S).

The construction of the parent antibody DNA sequence, encoding a humanised anti-Her2 receptor monoclonal antibody variant (trastuzumab) generated based on human IgG1(κ) framework, has previously been described in Carter P. et al. Proc. Natl Acad. Sci. USA, 89, 4285-4289 (1992), where the antibody is referred to as humAb4D5-8. For the purposes of the present experiments, the amino acids at positions 359 and 361 of the heavy chain amino acid sequence were replaced with Asp and Leu, respectively (E359D and M361L). The light and heavy chain amino acid sequences of the parent antibody used in the present experiments are also shown herein by SEQ ID NOs: 1 and 2, respectively. Two of the cysteines in the hinge region of the parent antibody (hinge region sequence: PKSCDKTHTCPPCP) form inter-chain disulfide bonds between the two heavy chains of the antibody. These cysteine residues correspond to positions 226 and 229 of IgG1 according to the EU-index numbering system, and are residues 229 and 232 of SEQ ID NO: 2.

The two engineered antibody variants (IgGC226S and IgGC229S) were created by PCR-based site-directed mutagenesis of the parent antibody heavy chain sequence to substitute one of the inter-heavy chain cysteine residues in the hinge region with the amino acid Ser. The PCR methodology used was primer overlapping extension, as described by Ho et al. Gene, 77 (1989) 51-59, to generate a modification in the hinge region sequence. PCR primer oligonucleotides were designed to incorporate nucleotide changes into the coding sequence of the subject antibody. In the Cys226Ser variant, the codon change was from TGC (Cys) to AGC (Ser). In the Cys229Ser variant, the codon change was from TGC (Cys) to AGT (Ser). The new sequence was cloned back into heavy chain expression vector, including other portions of the heavy chain. Final construct (after mutagenesis) was verified by full length sequencing of the insert.

The newly generated heavy chain construct was co-transfected with the corresponding light chain construct into HEK293 cells using polyethylenimine (PEI), expressed in a 6 day transient culture, and purified by a combination of Protein A and Size Exclusion Chromatography, based on the protocol from “Transient Expression in HEK293-EBNA1 Cells,” Chapter 12, in Expression Systems (eds. Dyson and Durocher). Scion Publishing Ltd., Oxfordshire, UK, 2007.

Conjugation of Bis-Sulfone-PEG(24)-val-cit-PAB-MMAE to Parent Antibody and Antibody Variants.

Conjugation of the antibody variants with 1, 1.5 or 2 equivalents of the polymeric conjugation reagent Bis-sulfone-PEG(24)-val-cit-PAB-MMAE per inter-chain disulfide bond was performed after antibody reduction. The reduction reactions were carried out at 4.7 mg/mL antibody concentration using 10 mM DTT for 1 h, at either 22° C. or 40° C. Buffer exchange was performed for each antibody variant to remove excess reductant. The polymeric conjugation reagent was prepared in 50% aq. acetonitrile at pH 8 immediately before conjugation. Antibody concentrations during conjugation were 3 mg/mL and the reactions were conducted overnight (16 h), at either 40° C. or 22° C. Reaction conditions for the conjugation reactions are summarised in Table 1 below:

TABLE 1
Conjugation conditions.

| | Reaction 1 | Reaction 2 | Reaction 3 | Reaction 4 | Reaction 5 | Reaction 6 |
|---|---|---|---|---|---|---|
| mAb | IgGC226S | IgGC226S | IgGC226S | IgGC226S | IgGC226S | IgGC226S |
| Reagent eq. | 1 eq. | 1.5 eq. | 2 eq. | 1 eq. | 1.5 eq. | 2 eq. |
| per S—S | | | | | | |
| Temp./° C. | 40 | 40 | 40 | 22 | 22 | 22 |
| | Reaction 7 | Reaction 8 | Reaction 9 | Reaction 10 | Reaction 11 | Reaction 12 |
| mAb | IgGC229S | IgGC229S | IgGC229S | IgGC229S | IgGC229S | IgGC229S |
| Reagent eq. | 1 eq. | 1.5 eq. | 2 eq. | 1 eq. | 1.5 eq. | 2 eq. |
| per S—S | | | | | | |
| Temp./° C. | 40 | 40 | 40 | 22 | 22 | 22 |

The “IgGC226S” variant has a Cys to Ser substitution at position 226, and thus a single inter heavy-chain disulfide bond at position 229. The “IgGC229S” variant has a Cys to Ser substitution at position 229, and thus a single inter heavy-chain disulfide bond at position 226.

After Buffer Exchange, Each Reaction was Analysed by Hydrophobic Interaction

Chromatography (HIC) to determine the stoichiometry of drug loading using % area of the peaks at 280 nm, as previously described. The average Drug to Antibody Ratio (DAR) and the drug conjugate species distribution (DAR 1-3) of the antibody-drug conjugates produced are shown in Table 2.

TABLE 2
Average DAR and species distribution for IgGC226S (reactions
1 to 6) and IgGC229S (reactions 7 to 12)-drug conjugates.

| Reaction | Average DAR | DAR 1-3 |
|---|---|---|
| 1 | 1.41 | 80% |
| 2 | 1.99 | 89% |
| 3 | 2.42 | 87% |
| 4 | 1.33 | 79% |
| 5 | 1.96 | 90% |
| 6 | 2.40 | 89% |
| 7 | 1.43 | 78% |
| 8 | 1.76 | 83% |
| 9 | 2.65 | 76% |
| 10 | 1.26 | 76% |
| 11 | 2.11 | 88% |
| 12 | 2.50 | 83% |

As shown by the data in Table 2, the process of the invention allows antibodies to be effectively conjugated at high levels of homogeneity, and with a low average DAR. Antibody-drug conjugates with low average DAR have a number of beneficial properties, including reduced clearance rate, higher therapeutic index and reduced toxicity than conjugates with higher average DAR.

### EXAMPLE 2

### Analysis of DAR Distribution

In Example 1, lowest average DAR for the single-hinge disulfide variants was obtained when using 1 equivalent of the polymeric conjugation reagent per inter-chain disulfide bond, both at 40° C. (reactions 1 and 7) and at 22° C. (reactions 4 and 10). To compare these results to those obtainable using the parent antibody, parent antibody was conjugated using 1 equivalent of the polymeric conjugation reagent per disulfide bond using the conditions set out in Example 1.

Average DAR for the parent antibody, IgGC226S and IgGC229S are shown in Table 3.

TABLE 3

| Conjugation Temp | Parent mAb | IgGC226S | IgGC229S |
|---|---|---|---|
| 40° C. | 1.91 | 1.41 | 1.43 |
| 22° C. | 1.89 | 1.33 | 1.26 |

As shown by the data in Table 3, the average DAR for the parent antibody was significantly higher than for the single-hinge disulfide variants IgGC226S and IgGC229S, at either 40° C. or at 22° C.

Distribution curves of the antibody-drug conjugate species produced by the conjugations reactions were also analysed to determine DAR distribution. In addition to lower average

DAR, it can be seen from FIG. 1 (conjugation at 40° C.) and FIG. 2 (conjugation at 22° C.) that the process of the invention yields antibody-drug conjugates (IgGC226S and IgGC229S) having reduced heterogeneity and improved yield than those produced using the parent antibody. Antibody-drug conjugates having improved homogeneity require less purification than mixtures of variable stoichiometry, and display reduced toxicity, and/or improved pharmacokinetics and thereby improved efficacy due to the absence of high drug-load species.

### EXAMPLE 3

### Conjugation of Bis-Sulfone-PEG(24)-val-cit-PAB-MMAE to Parent Antibody and Antibody Variant IgGC226S: Higher Retention of Interchain Bridging with IgGC226S

Conjugation of the parent antibody and the single-hinge disulfide variant IgGC226S with 1 molar equivalent of the conjugation reagent Bis-sulfone-PEG(24)-val-cit-PAB-MMAE per inter-chain disulfide bond was performed after antibody reduction (TCEP, 1 molar equivalent per interchain disulfide, 15 min, 40° C.). The conjugation reagent was prepared in DMSO (to give 5% (v/v) DMSO in reaction solution) immediately before conjugation. Antibody concentrations during conjugation were 4 mg/mL. Reactions were conducted overnight (16 h) at 40° C., after which time the reaction mixtures were treated with 10 mM DHA for 1 h at room temperature and then analysed by SDS-PAGE. The SDS-PAGE gels were stained with InstantBlue™ and imaged using an IMAGEQUANT™ LAS 4010 instrument (GE Healthcare) to determine the % of each species present within a lane. The SDS-PAGE results are shown in FIG. 3. In FIG. 3, the lanes labelled M show Novex Protein Standards (Invitrogen). Lanes 1 and 2 show the migration profiles of IgGC226S pre- and post-conjugation reaction respectively. Lanes 3 and 4 show the equivalent reactions for the parent antibody. When the heavy to heavy interchain disulfides of an antibody are not covalently bridged following conjugation, for example, due to disulfide bond scrambling, a band just below the 80 kDa marker of heavy-light chain dimer (H+L) is visible by SDS-PAGE. In contrast, when the heavy to heavy interchain disulfides are bridged following conjugation, a band just above the 160 kDa marker of antibody heavy-light chain tetramer (2H+2L) is visible. Comparing lanes 2 and 4, it can be seen that conjugating to IgGC226S, possessing a single inter heavy-chain disulfide, leads to a higher extent of bridging between the two heavy chains compared to the parent antibody, with two inter heavy-chain disulfides (80% vs 67% of antibody heavy-light chain tetramer respectively) and a lower extent of heavy-light chain dimer formation (17% v 31% respectively). The process of the invention thus improves the stability of the antibody conjugate by efficient bridging of the inter-heavy chain disulfide bond.

1. A process for preparation of an antibody conjugate comprising reacting an engineered antibody having a single inter-heavy chain disulfide bond with a conjugating reagent that forms a bridge between two cysteine residues derived from the disulfide bond.

2. A process as claimed in claim 1, wherein the antibody is an IgG1 or IgG4 molecule.

3. A process as claimed in claim 1, wherein the antibody is prepared by recombinant expression or chemical synthesis.

4. A process as claimed in claim 3, which comprises
a) mutating a nucleic acid sequence encoding a parent antibody, wherein said mutation results in deletion or substitution of one or more inter-heavy chain cysteine residues with an amino acid other than cysteine;
b) expressing the nucleic acid in an expression system; and
c) isolating the engineered antibody.

5. A process as claimed in claim 4, wherein site directed mutagenesis is carried out to incorporate nucleotide changes into the coding sequence of the parent antibody.

6. A process as claimed in claim 1, wherein said single inter-heavy chain disulfide bond is at position 226 or 229 of the antibody according to the EU-index numbering system.

7. A process as claimed in claim 1, wherein the antibody has a cysteine at position 226 and an amino acid other than cysteine at position 229 according to the EU-index numbering system, or an amino acid other than cysteine at position 226 and a cysteine at position 229 according to the EU-index numbering system.

8. A process as claimed in claim 7, wherein the amino acid other than cysteine does not include a thiol moiety, for example serine, threonine, valine, alanine, glycine, leucine or isoleucine, other polar amino acid, other naturally occurring amino acid, or non-naturally occurring amino acid.

9. A process as claimed in claim 1, wherein the antibody has a cysteine at position 226 and a serine at position 229 according to the EU-index numbering system, or a serine at position 226 and a cysteine at position 229 according to the EU-index numbering system.

10. A process as claimed in claim 1, wherein the antibody is an IgG1 molecule and comprises a sequence of Cys-Pro-Pro-Ser or Ser-Pro-Pro-Cys at positions 226-229 according to the EU-index numbering system; or is an IgG4 molecule and comprises a sequence of Cys-Pro-Ser-Ser or Ser-Pro-Ser-Cys at positions 226-229 according to the EU-index numbering system.

11. A process as claimed in claim 1, in which the single inter-heavy chain disulfide bond is in the hinge region of the antibody at a location different from a disulfide bond in the parent antibody.

12. A process as claimed in claim 1, wherein the reagent includes a diagnostic, therapeutic or labelling agent, or a binding agent capable of binding a diagnostic, therapeutic or labelling agent.

13. A process as claimed in claim 1, wherein the reagent includes a polymer.

14. A process as claimed in claim 1, in which the reagent contains the functional group: 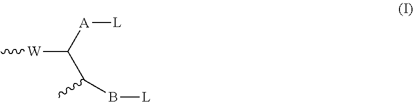
in which W represents an electron-withdrawing group; A represents a C₁₋₅ alkylene or alkenylene chain; B represents a bond or a C₁₋₄ alkylene or alkenylene chain; and each L independently represents a leaving group.

15. A process as claimed in claim 14, in which the reagent is of the formula II, III or IV: 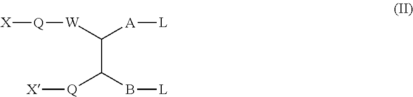
in which one of X and X′ represents a polymer and the other represents a hydrogen atom;
Q represents a linking group;
W represents an electron-withdrawing group; or, if X′ represents a polymer, X-Q-W together may represent an electron withdrawing group;
A represents a C₁₋₅ alkylene or alkenylene chain;
B represents a bond or a C₁₋₄ alkylene or alkenylene chain; and
each L independently represents a leaving group; 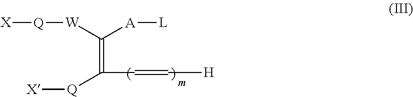
in which X, X′, Q, W, A and L have the meanings given for the general formula II, and in addition if X represents a polymer, X′ and electron-withdrawing group W together with the interjacent atoms may form a ring, and m represents an integer 1 to 4; or
X-Q-W—CR¹R^{1′}—CR².L.L′ (IV)
in which X, Q and W have the meanings given for the general formula II, and either
R¹ represents a hydrogen atom or a C₁₋₄alkyl group, R^{1′} represents a hydrogen atom, and each of L and L′ independently represents a leaving group; or
R¹ represents a hydrogen atom or a C₁₋₄alkyl group, L represents a leaving group, and R^{1′} and L′ together represent a bond; or
R¹ and L together represent a bond and R^{1′} and L′ together represent a bond; and
R² represents a hydrogen atom or a C₁₋₄ alkyl group.

16. A process as claimed in claim 14, which comprises the additional step of reducing the electron withdrawing group W in the resulting conjugate.

17. A process as claimed in claim 1, in which in addition to one molecule of conjugating reagent forming a bridge between the two cysteine residues derived from said inter-heavy chain disulfide bond, two additional molecules of conjugating reagent form bridges between the cysteine residues derived from the interchain disulfide bonds located between the C_{L} domain of the light chain and the C_{H}1 domain of the heavy chain of the antibody.

18. An antibody conjugate in which a conjugating reagent is bound to an antibody via two sulfur atoms derived from a disulfide bridge in the antibody; characterised in that said disulfide bridge is the only inter-heavy chain disulfide bond present in the antibody.

19. An antibody conjugate as claimed in claim 18, which additionally comprises two conjugating reagents bound via sulfur atoms derived the interchain disulfide bonds located between the CL domain of the light chain and the CH1 domain of the heavy chain of the antibody.

20. An antibody conjugate by a process as claimed in claim 1, wherein the conjugating reagent is bound to an antibody via

21. An antibody conjugate as claimed in claim 18, for use in therapy.

22. An antibody conjugate as claimed in claim 18, for use in treatment of cancer.

23. The use of an antibody conjugate as claimed in claim 18, for manufacture of a medicament for treatment of cancer.

24. A method of treating cancer which comprises the administration to a patient of an antibody conjugate as claimed in claim 18.

25. A pharmaceutical composition comprising a conjugate as claimed in claim 18, together with a pharmaceutically acceptable carrier; optionally together with an additional therapeutic agent.
